(19) United States
(12) Patent Application Publication
(10) Pub. No.: US 2004/0059309 A1
(43) Pub. Date: Mar. 25, 2004

(54) Absorbent article with untreated hydrophobic target area

(76) Inventor: Brian Keith Nortman, Appleton, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/246,034

(22) Filed: Sep. 18, 2002

(51) Int. Cl. A61F 13/15; A61F 13/20

(52) U.S. Cl. 604/378

(57) ABSTRACT

An incontinence garment or other absorbent article may have improved transepidermal water loss values resulting in higher skin dryness for the wearer. The garment further retains a degree of run-off control without adversely affecting leakage from the article. The target zone of the article liner, or the area most likely to receive surging insults of urine, is hydrophobic, i.e. is left untreated in its original hydrophobic state or rendered more hydrophobic after a hydrophilic surfactant treatment. The target zone is surrounded by areas treated to be hydrophilic. These treated areas are typically the leg and waist areas or margins of a garment. Thus an economical article delivering improved wearer comfort may be manufactured.

### BACKGROUND OF THE INVENTION

Personal products, or absorbent articles, including incontinence garments such as limited use or disposable diapers and adult incontinence garments; and sanitary pads often are made with a liner material, also referred to as the top sheet; an absorbent core, and a liquid impervious back sheet, or outer layer. Some such items may also have a surge layer for fluid uptake and distribution, or other specialized layers between the top sheet and absorbent core. Absorption and retention of fluid, comfort, and avoidance of leakage are the functions desired of such products.

Typically, the absorbent core is the primary liquid retention layer and includes a web of fibers loosely holding an amount of absorbents or superabsorbents, or both. The web of fibers provides structure for the absorbent core and optionally contributes to absorbency. Superabsorbent particles are added to the web to increase the absorbency and retention effectiveness of the webs on a unit mass basis.

As known in the art, typically, the liner web, or material, facing the wearer of an incontinence garment is a skin-friendly, i.e. good hand, low basis weight, nonwoven web of spunbond thermoplastic fibers, such as polypropylene, which is hydrophobic in nature. The liner material is then rendered hydrophilic over its entire surface, or at least a three inch wide zone on the longitudinal midline of the liner, by a surfactant treatment of the fibers, such as by application of AHCOVEL treatment (available from Hodgson Chemicals, USA) or mixtures of AHCOVEL treatment and GLUCOPON treatment (available from Cognis, North America) to provide less surface run-off of urine and a higher rate of urine pass-through to the absorbent core, i.e., the primary liquid retention layer, of the garment. The pass-through to the absorbent core may be aided by a so-called “surge” layer, between the liner and the absorbent core, which is primarily a liquid uptake and distribution material layer, as will be understood by the person having ordinary skill in the art. Unfortunately, although the run-off of urine is decreased by the presence of fibers treated to be hydrophilic, the hydrophilicity of the fibers allows the liner material, which is next to the skin, to remain damp, thereby increasing wearer discomfort. It may therefore be desirable to provide a liner with less emphasis on run-off control while retaining a dry target zone, i.e. that area most likely to receive urine insults, to increase wearer comfort.

U.S. Pat. No. 6,183,847 to Goldwasser teaches that a nonwoven material having meltblown fibers, such as a so-called SMS (spunbond-meltblown-spunbond) multi-component material, i.e., typically a fluid barrier material within the context of absorbent articles, may be treated with surfactants to be rendered hydrophilic or fluid permeable so as to remove some of the barrier properties of the meltblown-containing nonwoven. Alternatively, Goldwasser suggests that a meltblown-containing nonwoven exhibiting some hydrophilic characteristics may be made more hydrophobic by use of the surfactant coating process therein, apparently to be made more suitable for barrier applications. It is uncertain what the effect of the Goldwasser teaching would have on the overall skin dryness of a wearer of an incontinence garment since this subject is not addressed in the teaching.

It is believed that there exists a further need in the art for an economical and efficacious method of rendering the target area of a personal product such as an incontinence garment, e.g., a diaper, training pant, or adult incontinence garment; less susceptible to urine run-off and more comfortable to the wearer by decreasing the TransEpidermal Water Loss (TEWL) value of the garment, which is a measure of quantitative skin dryness.

### SUMMARY OF THE INVENTION

The present invention provides solutions to the above-described need in its various aspects. By way of general summary, the present invention may provide a specially constructed liner, or top sheet, in order to enhance the utility of the absorbent article.

It has been found that an incontinence garment may have improved TEWL values (i.e. higher skin dryness) when the target zone of the garment liner, or the area most likely to receive surging insults of urine, is left untreated, i.e., in its original hydrophobic state, and then having the target area surrounded by the areas treated to be hydrophilic in order to retain some degree of surface run-off control in the liner area. These treated areas are typically the leg and waist areas or margins of the garment.

In certain aspects of the present invention a liner may be produced from a web of hydrophobic fibers. The liner web is then treated in those areas referenced to the completed diaper, so as to render only the area surrounding the target zone hydrophilic, while leaving the target zone hydrophobic. In examining the problem of a hydrophilic target zone, the inventor has determined that leaving the target zone hydrophobic according to the present invention results in a greater wearer comfort as measured by TEWL values, while not resulting in adversely increased leakage of exudates from the diaper.

A number of plausible reasons may help to explain this phenomenon. Without wishing to be bound by theory, it is believed that actual use of a garment may result in liquid insults which are propelled through the untreated liner because of more intimate body-to-garment contact, higher pressure of fluid flow, and the like. Further, modern incontinence garment construction has provided elasticized cuff areas, containment flaps, and the like, which further reduce the chance of leakage and necessary dependence upon a highly hydrophilic liner layer.

In certain aspects of the present invention, a liner may be produced from a web of hydrophobic thermoplastic, e.g. polypropylene, spunbond fibers which are then treated by a printing or spraying process, so as to render only the area surrounding the target zone hydrophilic in those areas referenced to the completed diaper, while leaving the target zone hydrophobic.

In certain other aspects of the present invention, a liner may be produced from a web of hydrophobic thermoplastic, e.g. polypropylene, spunbond fibers which are then treated, in those areas referenced to the completed diaper, so as to render hydrophilic selected areas of the target zone and areas surrounding the target zone, while leaving effective areas or placements of hydrophobic liner for the target zone. Examples of surfactant application techniques may include, but are not necessarily limited to: a printing or spraying process, including but not limited to, relief, intaglio, planographic, and spray printing methods such as bubble jet or ink jet methods.

In certain other aspects of the present invention, a liner may be produced from a web of hydrophobic thermoplastic fibers and be rendered hydrophilic by application of surfactant by printing or by any of various coating processes such as roller, brush, immersion, or foam processes as known in the art, or their equivalents. The target zone, or area, may then be subsequently be rendered hydrophobic in relation to the initial liner coating by additional treatments.

In certain other aspects of the present invention, a hydrophobic target area liner may be economically produced according to invention and fitted within a completed absorbent article to improve the comfort of the absorbent article while maintaining a degree of run-off control and/or leakage performance for the finished article.

### BRIEF DESCRIPTION OF THE DRAWINGS

The accompanying drawings are presented as an aid to explanation and understanding of various aspects of the present invention only and are not to be taken as limiting the present invention. The drawings are not necessarily to scale, nor should they be taken as photographically accurate depictions of real objects unless otherwise stated.

FIG. 1 illustrates a garment according to the present invention, in this case an exemplary diaper.

FIGS. 2-8 illustrate hydrophilic treatment patterns applicable to areas surrounding or interfacing with the target zone of a liner.

FIG. 9 illustrates a dual layer liner used with particular embodiments to empirically test aspects of the present invention.

### DEFINITIONS

“Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, it is generally considered that fibers having contact angles less than 90 degrees are designated “wettable” or hydrophilic, while fibers having contact angles greater than 90 degrees are designated “nonwettable” or hydrophobic.

A “layer” is defined as a generally recognizable combination of similar material types or function existing in the X-Y plane.

As used herein the term “microfibers” means small diameter fibers having an average diameter not greater than about 75 microns (μm), for example, having an average diameter of from about 0.5 microns (μm) to about 50 microns (μm), or more particularly, microfibers may have an average diameter of from about 2 microns (μm) to about 40 microns (μm). Another frequently used expression of fiber diameter is denier, which is defined as grams per 9000 meters of a fiber and may be calculated as fiber diameter in microns (μm) squared, multiplied by the density in grams/cc, multiplied by 0.00707. A lower denier indicates a finer fiber and a higher denier indicates a thicker or heavier fiber. For example, the diameter of a polypropylene fiber given as 15 microns (μm) may be converted to denier by squaring, multiplying the result by 0.89 g/cc and multiplying by 0.00707. Thus, a 15 micron (μm) polypropylene fiber has a denier of about 1.42 (152×0.89×0.00707=1.415). Outside the United States the unit of measurement is more commonly the “tex”, which is defined as the grams per kilometer of fiber. Tex may be calculated as denier/9.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface.

“Nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Personal Products” shall include: absorbent articles used to absorb any fluid including human body fluids, such as diapers, adult incontinence garments, training pants, absorbent swim pants, feminine care products, hygienic wipes, absorbent pads and the like; disposable tissue products for personal use, such as bath tissue, facial tissue, paper towels and napkins; disposable apparel for institutional, industrial and consumer use; disposable health care products that are not intended to be cleaned for reuse, such as caps, gowns, foot wear, masks, drapes, wraps, covers, and the like; consumer health care products; and health care or environmental diagnostic devices that are at least partially disposable.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as taught, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al. Spunbond fibers are quenched and generally not tacky when they are deposited onto a collecting surface. Spunbond fibers are generally continuous and often have average deniers larger than about 0.3, more particularly, between about 0.6 and 10.

Words of degree, such as “about”, “substantially”, and the like are used herein in the sense of “at, or nearly at, when given the manufacturing and material tolerances inherent in the stated circumstances” and are used to prevent the unscrupulous infringer from unfairly taking advantage of the invention disclosure where exact or absolute figures are stated as an aid to understanding the invention.

As used herein, the term “consisting essentially of” does not exclude the presence of additional materials which do not significantly affect the desired characteristics of a given composition or product. Exemplary materials of this sort would include, without limitation, pigments, antioxidants, stabilizers, surfactants, waxes, flow promoters, solvents, particulates, and materials added to enhance processability of the composition.

### DETAILED DESCRIPTION

The various aspects and embodiments of the invention will be described in the context of disposable absorbent articles, and more particularly referred to, without limitation and by way of illustration only, as a disposable diaper. It is, however, readily apparent that the present invention could also be employed to produce other products or garments, such as feminine care articles, various incontinence garments, medical garments and any other disposable garments. Typically, the disposable garments are intended for limited use and are not intended to be laundered or otherwise cleaned for reuse. A disposable diaper, for example, is discarded after it has become soiled by the wearer.

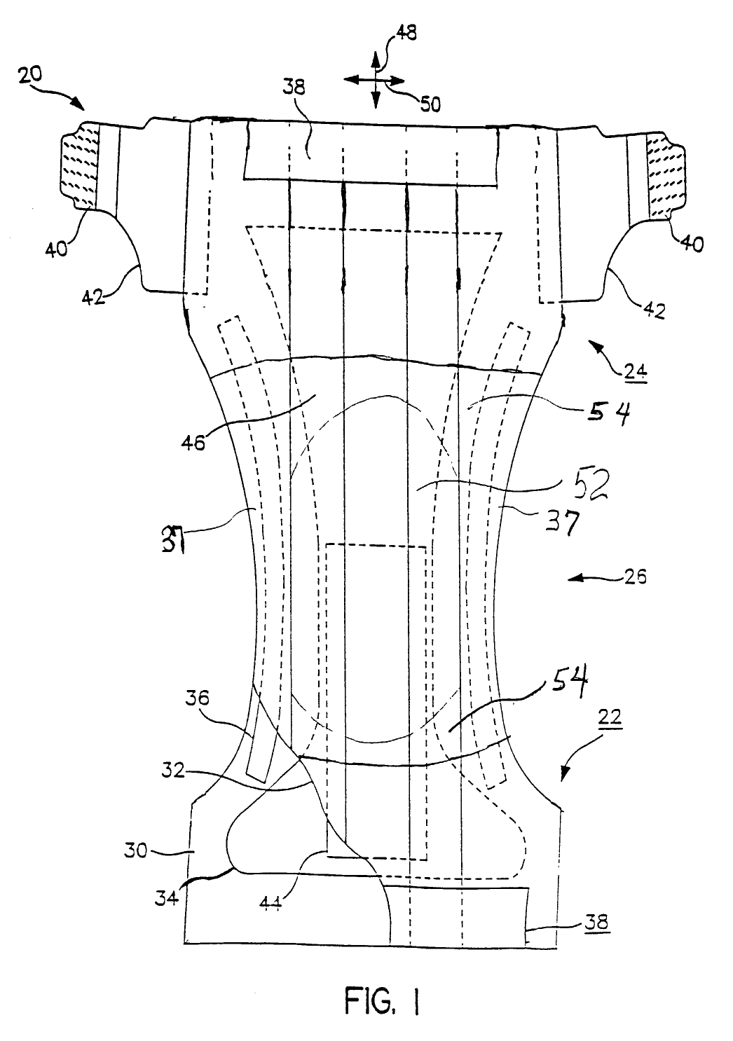

FIG. 1 is a representative plan view of an absorbent article, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs. Thus, the intermediate section 26 generally includes the target zone, or area where repeated liquid surges, or insults, typically occur in the diaper.

The diaper 20 includes, without limitation, an outer cover, or back sheet 30, a liquid permeable bodyside liner, or topsheet, 32 positioned in facing relation with the back sheet 30, and an absorbent core, or body, being the primary liquid retention structure, 34, such as an absorbent pad, which is located between the back sheet 30 and the liner 32. The liner 32 is indicated as having a hydrophobic area 52 surrounded by hydrophilic areas 54. As further explained below, the hydrophobic area 52 may represent an unbroken area of hydrophobic fibers, or may include noncontiguous areas representing a total area effective to achieve an efficacious level of wearer dryness. The hydrophilic areas 54, while indicated as surrounding the hydrophobic area 52 at all margins of the diaper 20, may not necessarily do so in all embodiments. The liner material is typically a spunbond laminate of about 0.5 osy basis weight of polypropylene thermoplastic fibers made into a breathable and liquid permeable nonwoven web.

The back sheet 30 defines a length, or longitudinal direction 48, and a width, or lateral direction 50 which, in the illustrated embodiment, coincide with the length and width of the diaper 20. The liquid retention structure 34 generally has a length and width that are less than the length and width of the back sheet 30, respectively. Thus, marginal portions of the diaper 20, such as marginal sections of the back sheet 30, may extend past the terminal edges of the liquid retention structure 34. In the illustrated embodiments, for example, the back sheet 30 extends outwardly beyond the terminal marginal edges of the liquid retention structure 34 to form side margins and end margins of the diaper 20. The liner 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands in the leg margin areas, collectively 37, which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer.

In the illustrated embodiment, the diaper 20 includes a pair of side panels 42 to which fasteners 40, indicated as the hook portion of a hook and loop fastener, are attached. Generally, the side panels 42 are attached to the side edges of the diaper 20 in one of the waist sections 22, 24 and extend laterally outward therefrom. The side panels 42 may be elasticized or otherwise rendered elastomeric. For example, the side panels 42, or indeed, any precursor component webs of the garment, may be an elastomeric material such as a neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al. Examples of absorbent articles that include elasticized side panels and selectively configured fastener tabs are described in PCT Patent Application No. WO 95/16425 published Jun. 22, 1995 to Roessler; U.S. Pat. No. 5,399,219 issued Mar. 21, 1995 to Roessler et al.; U.S. Pat. No. 5,540,796 to Fries; U.S. Pat. No. 5,595,618 to Fries and U.S. Pat. No. 5,496,298 to Kuepper et al.

The diaper 20 may also include a surge management layer 44, located between the liner 32 and the liquid retention structure 34, to rapidly accept fluid exudates and distribute the fluid exudates to the liquid retention structure 34 within the diaper 20. The diaper 20 may further include a ventilation layer (not illustrated) located between the liquid retention structure 34 and the back sheet 30 to insulate the back sheet 30 from the liquid retention structure 34 to reduce the dampness of the garment at the exterior surface of the back sheet 30. Examples of suitable surge management layers 44 are described in U.S. Pat. No. 5,486,166 to Bishop and U.S. Pat. No. 5,490,846 to Ellis.

As illustrated in FIG. 1, the exemplary disposable diaper 20 may also include a pair of containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

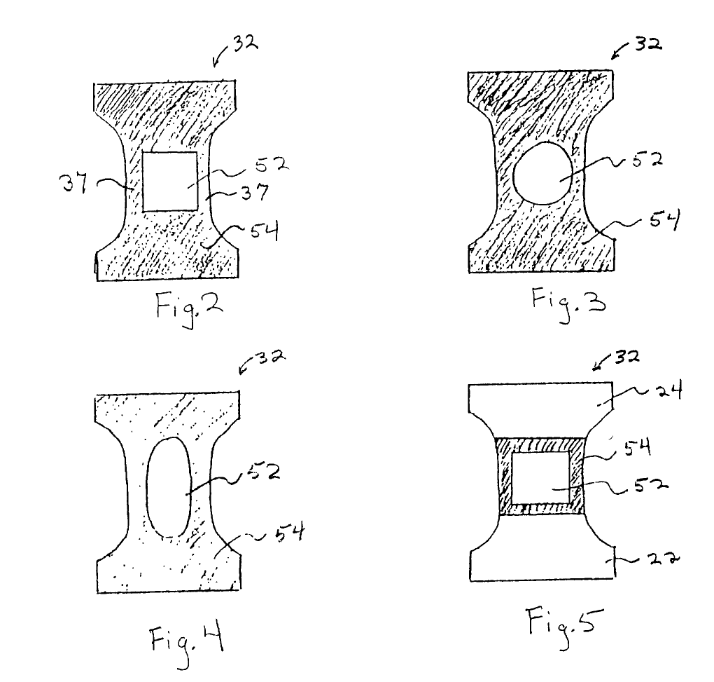

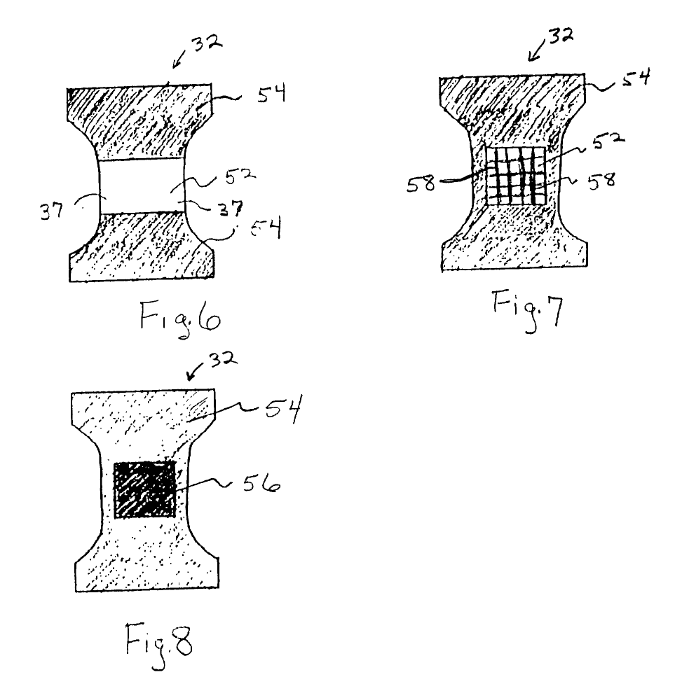

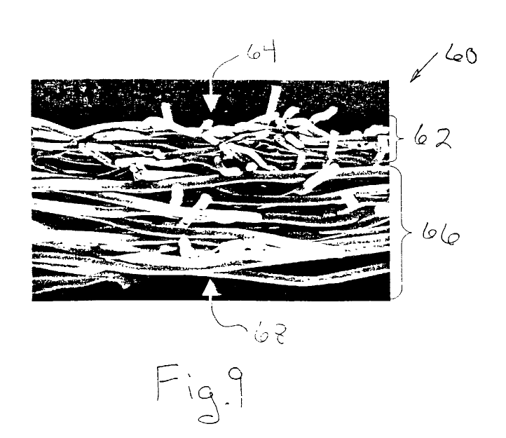

Referencing FIGS. 2-8, a diaper 20 is shown in basic schematic outline with the liner 32 facing the viewer. The cross hatch area represents the treated area 54 made to be more hydrophilic than the original hydrophobic liner material. FIG. 2 illustrates a liner 32 with a central untreated hydrophobic area 52 corresponding to the target area in the shape of a rectangle, while the remaining area of the liner 32 is a treated hydrophilic area, including the areas of the leg margins, collectively 37. A target area rectangle will generally be at least about three inches by about three inches but will, of course, be dependent upon the application for which the product is designed. FIG. 3 illustrates an embodiment similar to FIG. 2 but having a circular untreated hydrophobic area 52. FIG. 4 illustrates an embodiment similar to FIG. 2 but having an elongated oval shaped untreated hydrophobic area 52. FIG. 5 illustrates an embodiment similar to FIG. 2 but having a untreated hydrophobic area 52 in the target area and additional untreated hydrophobic areas in the front waist section 22 and the rear waist section 24, leaving the treated area 54 as a border around the untreated hydrophobic area 52 in the intermediate area 26 (FIG. 1) of the diaper. FIG. 6 illustrates a liner 32 with a central untreated hydrophobic area 52 corresponding to the target area in the shape of a rectangle across the transverse, or lateral, width of the diaper, including the areas of the leg margins, collectively 37. FIG. 7 illustrates a liner 32 with a central untreated hydrophobic area 52 corresponding to the target area in the shape of a rectangle including hydrophilic treated stripes 58 within the central untreated hydrophobic area 52. FIG. 8 illustrates a liner 32 with a central hydrophobic area 56 corresponding to the target area in the shape of a rectangle which has been treated to render the target area hydrophobic after the entire liner has been treated to be hydrophilic. Such hydrophilic treatment may include topical applications or internal use of surfactant at the fiber level. The treated hydrophobic area 56 is surrounded over the rest of the surface by a hydrophilic treated area 54.

The application of surfactants to provide the treated area 54 as a border around the untreated hydrophobic area 52 can be accomplished in various manners as befits the manufacturing facility, type of materials in use, and other practical or aesthetic considerations and is left to the discretion of the person having ordinary skill in the art. The desired application may be accomplished, without limitation, through such means as arrangement of the applicators, e.g., spray heads; intermittent application of spray through timed control of the applicators, roller printing techniques, or combinations thereof.

As can be seen from Table 1, three diaper types with completely untreated, i.e. hydrophobic, liners generally have lower TEWL values than control diapers of the same construction but with treated, i.e. hydrophilic, liners. Generally, each diaper type a-c included a Dual Layer Liner (DLL) 60, as seen in FIG. 9, which has a two layer 0.5 or 0.6 osy polypropylene web, point bonded over between about 15-21% of its area, with a first 2.5 denier per fiber (dpf) layer 62 on the body side 64 and a second 5.0 dpf layer 66 distally located 68 from the body side, at 33% and 67% by weight, respectively. The second layer 66 may be surfactant treated to be hydrophilic with AHCOVEL (available from Hodgson Chemicals, USA) or mixtures of AHCOVEL and GLUCOPON (available from Cognis, North America). The liners, whether the DLL of the examples, or a single layer liner which is envisioned to be a more common embodiment of the present invention, can be treated with a solution in the proportions of 42 g of AHCOVEL Base N-62 surfactant, which is a blend of about 50 weight percent sorbitan mono-oleate and about 50 weight percent hydrogenated ethoxylated castor oil at 100 percent solids supplied by ICI Chemicals; 4.6 g GLUCOPON UP-220, an alkyl polygly-coside with a C8-10 chain at 60 percent solids supplied by Henkel Chemicals; and 40 g hexanol. The DLL is adhesive bonded to a surge layer comprising a through air bonded carded web of mixed bicomponent polyolefin (polyethylene/polypropylene or polyethylene/polyethylene terephthalate) and PET (polyethylene terephthalate) staple fibers, of between about 2.5 osy to about 3.0 osy. The outer cover, or backsheet, was a commercial, breathable outer cover (e.g., about a 12,000 MOCON water vapor transmission rate value) comprising a laminate of microporous film and spunbond nonwoven facing material. For a description of MOCON water vapor transmission rate value the reader is referred to U.S. Pat. No. 6,414,217 to Uitenbroek et al.

Test diaper types comprised the following parts:

Diaper a included a liner of 0.5 osy dual layer liner (DLL), a surge layer of 2.5 osy, and a liquid retention structure of about 14 g of superabsorbent material and about 20 g of fluff. A tissue layer of about 17 gsm separated the surge layer from the liquid retention structure.

Diaper b included a liner of 0.5 osy dual layer liner (DLL), a surge layer of 2.5 osy, and a liquid retention structure of about 14 g of superabsorbent material and about 20 g of fluff. A tissue layer of about 17 gsm separated the surge layer from the liquid retention structure.

Diaper c included a liner of 0.6 osy dual layer liner (DLL), a surge layer of 3.0 osy, and a liquid retention structure of about 10.9 g of superabsorbent material and about 14.8 g of fluff. A tissue layer of about 21 gsm separated the surge layer from the liquid retention structure.

TABLE 1

| | % Leakage | | TEWL | | | Run-off | |
|---|---|---|---|---|---|---|---|
| Diaper | Un- | | Un- | | | Un- | |
| type | treated | treated | treated | treated | Δ | treated | treated |
| a | 4.7 | 4.0 | 20.9 | 22.1 | −1.2 | 53 | 0 |
| b | 4.1 | 4.2 | 17.5 | 19.2 | −1.7 | 50 | 0 |
| c | 5.6 | 6.5 | 17.4 | 20.8 | −3.4 | 70 | 23 |

Discussion of Results

The percentage of diapers subject to leakage under direct observation in confidential field testing of diaper lots between about 1500 and 2000 samples shows no statistically significant differences at the 95% confidence level between treated and untreated diapers. Importantly, the three diaper types show a significant improvement of over 1.0 TEWL points for an untreated diaper liner over that of a traditionally treated hydrophilic liner. As will be noted, run-off from the untreated liners was significantly higher than for treated liners. Thus, the embodiments of the present invention call for a liner comprising both the hydrophobic target area and a hydrophilic area in order to reduce anticipated run-off values in the finished product.

### Test Methods

TEWL Testing

1. An adult female subject must test one of each different armband code during the study.

2. The subjects report to an environmentally controlled room. The subjects wait quietly in the environmentally controlled room fifteen minutes prior to testing allowing them to acclimate to environmental conditions of 73 degrees Fahrenheit and 50% relative humidity.

3. The subject extends and rests her arm(s) out on a table with the inner forearm facing up, one arm at a time.

4. The study conductor will place an evaporimeter probe on the subject's inner mid forearm area, making certain that the probe is held securely on the forearm.

5. The evaporimeter will collect a two minute TEWL baseline reading from the subject's inner mid forearms.

Note: If the average TEWL baseline reading is over 10 g/m2/hr the subject will need to wait another ten to fifteen minutes and have the TEWL readings repeated. If the average TEWL baseline reading is 10 g/m2/hr or below, that reading is recorded. Subjects who continue to have high baseline readings will be discontinued from the study.

6. After the initial TEWL reading(s) have been obtained the study conductor places a plastic connector, connected to rubber tubing, at the TEWL reading site with the opening of the plastic connector facing out.

7. The study conductor wraps the appropriate test sample around the subject's forearm(s) with the target area of the test sample over the opening of the plastic connector. The test sample should not overlap at the target area.

8. The study conductor secures the test sample in place by fastening masking tape on the upper and lower portions of the test sample making sure the tape does not come in contact with the subject's skin and does not go over the target area.

9. The subject will hold her arm(s) out with the inner forearm(s) facing down.

10. The study conductor administers three of either a 60 ml or a 70 ml insult(s) of 0.9% saline with a 45 sec delay between fills, through the rubber tubing to the test sample.

11. The study conductor removes the plastic connector and tubing from under the test sample.

12. The study conductor places an elastic retainer, such as a nylon netting, over the entire test sample to hold it securely on the forearm.

13. The subject wears the test sample for a total of sixty minutes.

14. The subject waits quietly in the environmentally controlled room during the sixty minute wear time.

15. The study conductor removes the elastic retainer(s) and test sample after wear time.

16. The study conductor collects final TEWL reading(s) with the evaporimeter.

17. The TEWL value is normalized by subtracting the baseline TEWL value from the post-diaper TEWL value.

A felt tip pen can be employed to mark an “X” at the target zone inside the diaper, with the “X” positioned appropriately, such as about five inches below the top front edge of the absorbent pad and centered side-to-side. The TEWL measurements are taken with an evaporimeter, such as an Evaporimeter EPI instrument distributed by Servomed AB, Stockholm, Sweden, or a Dermalab evaporimeter from Cortex Technology of Denmark. Each test measurement is taken over a period of two minutes with TEWL values taken once per second (a total of 120 TEWL values). The digital output from the evaporimeter instrument gives the rate of transepidermal water loss (TEWL) in g/m²/hr.

Run-Off Testing

This test is also sometimes called a liner wettability test. Values are expressed as the number of millimeters (ml) out of 100 ml of distilled or deionized water at 35° C.±1.7° C. that runs off about a 200 mm length of the product sample, at standard laboratory atmosphere, when placed and held on a 30 degree incline and the 100 ml of water is released at a height of 10 mm above the sample through a stopcock. Flow rate of water should be 100 ml/15 sec. ±1.5 sec. When a garment is used as the product sample, all elastics are to be trimmed in order that the garment may lay flat on the incline board.

Leakage Testing

Sample diapers were distributed under confidentiality provisions for experimental testing to about 200 caregivers for use on infants. The caregivers recorded incidents of leakage through the sample diapers.

While the invention has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

We claim:

1. A process of making an incontinence garment, the incontinence garment having a target area most likely to receive urine insults, comprising the steps of:
a) providing a liner including a hydrophobic nonwoven web;
b) treating a first surface area of the liner, corresponding to a non-target area of the liner, so as to be rendered hydrophilic;
c) creating a hydrophobic second area of the liner, corresponding to the target area of the liner;
d) providing a backsheet layer for the incontinence garment;
e) providing a liquid retention layer for the incontinence garment; and
f) joining the liquid retention layer between the backsheet layer and the liner so as to incorporate the liner into the incontinence garment with the second area placed in the target area of the incontinence garment.

2. The process of claim 1 wherein the hydrophobic area is provided by not treating the second area thereby leaving the second area in its substantially original hydrophobic condition.

3. The process of claim 1 wherein the hydrophobic area is provided by additionally treating the second area to render the second area more hydrophobic than the first area.

4. A process of making an incontinence garment, the incontinence garment having a target area most likely to receive urine insults, comprising the steps of:
a) providing a liner including a hydrophobic nonwoven web comprising thermoplastic spunbond fibers;
b) treating a first surface area of the liner, corresponding to a non-target area of the liner, so as to be rendered hydrophilic;
c) leaving a second area of the liner, corresponding to the target area of the liner, untreated in its substantially original hydrophobic condition;
d) providing a backsheet layer for the incontinence garment;
e) providing a liquid retention layer for the incontinence garment; and
f) joining the liquid retention layer between the backsheet layer and the liner so as to incorporate the liner into the incontinence garment with the second area placed in the target area of the incontinence garment.

5. The process of claim 4 further comprising the step of supplying a surge layer between the liner and the liquid retention layer.

6. The process of claim 5 wherein the surge layer comprises a mixture of polyolefin bicomponent fibers and polyester staple fibers in a bonded carded web.

7. The process of claim 4 wherein the untreated second area is an area effective to produce a reduction in transepidermal water loss over a similar diaper construction having no untreated second area in the liner.

8. The process of claim 4 wherein the treated first area is rendered hydrophilic by treating it with a liquid surfactant.

9. The process of claim 8 wherein the treated first area is rendered hydrophilic by treating it with a solution in the proportions of 42 g of a surfactant being a blend of about 50 weight percent sorbitan mono-oleate and about 50 weight percent hydrogenated ethoxylated castor oil at 100 percent solids; 4.6 g of an alkyl polygly-coside with a C8-10 chain at 60 percent solids; and 40 g hexanol.

10. The process of claim 4 wherein the treated first area is rendered hydrophilic by placing surfactants thereon with printing methods.

11. The process of claim 4 wherein the treated first area is rendered hydrophilic by placing surfactants thereon with coating methods.

12. The process of claim 4 wherein the untreated second area is at least about three inches by about three inches.

13. The process of claim 4 wherein the liner comprises polypropylene fibers.

14. The process of claim 4 wherein the liner comprises a first layer of smaller denier polypropylene fibers and a second layer of larger denier polypropylene fibers.

15. The process of claim 14 wherein the layer of larger denier polypropylene fibers is positioned proximal the liquid retention layer and is treated to be rendered hydrophilic.

16. An incontinence garment having a target area most likely to receive urine insults, comprising:
a) a liner including a hydrophobic nonwoven web; a first surface area, corresponding to a non-target area of the liner, treated so as to be rendered hydrophilic; and a hydrophobic second area, corresponding to the target area of the liner,
b) a backsheet layer for the incontinence garment;
c) a liquid retention layer for the incontinence garment; and
d) the liquid retention layer positioned between the backsheet layer and the liner so as to incorporate the liner into the incontinence garment with the hydrophobic second area placed in the target area of the incontinence garment.

17. The incontinence garment of claim 16 wherein the hydrophobic second area is left untreated in its substantially original hydrophobic condition.

18. The incontinence garment of claim 16 wherein the hydrophobic second area contains a surfactant to render the second area more hydrophobic than the first area.

19. The incontinence garment of claim 16 further comprising a surge layer between the liner and the liquid retention layer.

20. The incontinence garment of claim 19 wherein the surge layer comprises a mixture of polyolefin bicomponent fibers and polyester staple fibers in a bonded carded web.

21. The incontinence garment of claim 16 wherein the hydrophobic second area is an area effective to produce a reduction in transepidermal water loss over a similar diaper construction having no hydrophobic second area in the liner.

22. The incontinence garment of claim 16 wherein the hydrophilic first area is treated with a liquid surfactant.

23. The incontinence garment of claim 16 wherein the hydrophilic first area is treated with a solution in the proportions of 42 g of a surfactant being a blend of about 50 weight percent sorbitan mono-oleate and about 50 weight percent hydrogenated ethoxylated castor oil at 100 percent solids; 4.6 g of an alkyl polygly-coside with a C8-10 chain at 60 percent solids; and 40 g hexanol.

24. The incontinence garment of claim 16 wherein the hydrophobic target area is at least about three inches by about three inches.

25. The incontinence garment of claim 16 wherein the liner comprises polypropylene fibers.

26. The incontinence garment of claim 25 wherein the liner comprises spunbond polypropylene fibers.

27. The incontinence garment of claim 16 wherein the liner comprises a layer of smaller denier polypropylene fibers and a layer of larger denier polypropylene fibers.

28. The incontinence garment of claim 27 wherein the layer of larger denier polypropylene fibers is positioned proximal the liquid retention layer and is treated to be rendered hydrophilic.

29. An incontinence garment having a target area most likely to receive urine insults, comprising:
a) a liner including a hydrophobic nonwoven web comprising thermoplastic spunbond fibers;
b) a first surface area, corresponding to a non-target area of the liner, treated so as to be rendered hydrophilic;
c) a second area, corresponding to the target area of the liner, left untreated in its substantially original hydrophobic condition;
d) a backsheet layer for the incontinence garment;
e) a liquid retention layer for the incontinence garment;
f) the liquid retention layer joined between the backsheet layer and the liner so as to incorporate the liner into the incontinence garment with the second area placed in the target area of the incontinence garment; and
g) a surge layer between the liner and the liquid retention layer.

30. The incontinence garment of claim 29 wherein the surge layer comprises a mixture of polyolefin bicomponent fibers and polyester staple fibers in a bonded carded web.

31. The incontinence garment of claim 30 wherein the untreated second area is an area effective to produce a reduction in transepidermal water loss over a similar incontinence garment construction having no hydrophobic second area in the liner.

32. The incontinence garment of claim 31 wherein the treated first area is rendered hydrophilic by treating it with a liquid surfactant.

33. The incontinence garment of claim 32 wherein the treated first area is rendered hydrophilic by treating it with a solution in the proportions of 42 g of a surfactant being a blend of about 50 weight percent sorbitan mono-oleate and about 50 weight percent hydrogenated ethoxylated castor oil at 100 percent solids; 4.6 g of an alkyl polygly-coside with a C8-10 chain at 60 percent solids; and 40 g hexanol.

34. The incontinence garment of claim 32 wherein the untreated second area is at least about three inches by about three inches.

35. The incontinence garment of claim 34 wherein the liner consists essentially of polypropylene fibers.
